(19) United States
(12) Patent Application Publication
(10) Pub. No.: US 2019/0192077 A1
(43) Pub. Date: Jun. 27, 2019

(54) System and method for extracting and analyzing in-ear electrical signals

(71) Applicant: 10115045 Canada Inc., Vancouver (CA)

(72) Inventors: Sean Kaiser, Vancouver (CA); Kristina Pearkes, Vancouver (CA)

(21) Appl. No.: 16/213,116

(22) Filed: Dec. 7, 2018

Related U.S. Application Data
(60) Provisional application No. 62/595,952, filed on Dec. 7, 2017.

(51) Int. Cl. A61B 5/00; A61B 5/0478

(52) U.S. Cl. CPC A61B 5/6817; A61B 5/16; A61B 5/0478

(57) ABSTRACT

A system for sensing in-ear electrical signals includes a left electrode tip, a right electrode tip, and a signal acquisition subsystem. Each electrode tip includes: an elastic substrate configured to conform against an internal surface of an ear canal of a user; a sense electrode, a reference electrode, and a driven ground electrode each arranged on the outer surface of the elastic substrate. The signal acquisition subsystem is configured to, during a sampling period: output a left time series of a left voltage differential between the left sense electrode and the right reference electrode; and output a right time series of a right voltage differential between the right sense electrode and the left reference electrode.

### CROSS-REFERENCE TO RELATED APPLICATIONS

This Application claims the benefit of U.S. Provisional Application No. 62/595,952, filed on 7 Dec. 2017, which is incorporated in its entirety by this reference.

### TECHNICAL FIELD

This invention relates generally to the field of brain computer interfaces and more specifically to a new and useful method for extracting and analyzing electrical activity from inside the ear canal in the field of brain computer interfaces.

### BRIEF DESCRIPTION OF THE FIGURES

FIG. 1 is a schematic representation of a system;

FIG. 2 is a flowchart representation of a method;

FIG. 3 is a schematic representation of a first variation of the system; and

FIG. 4 is a schematic representation of a second variation of the system.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. SYSTEM

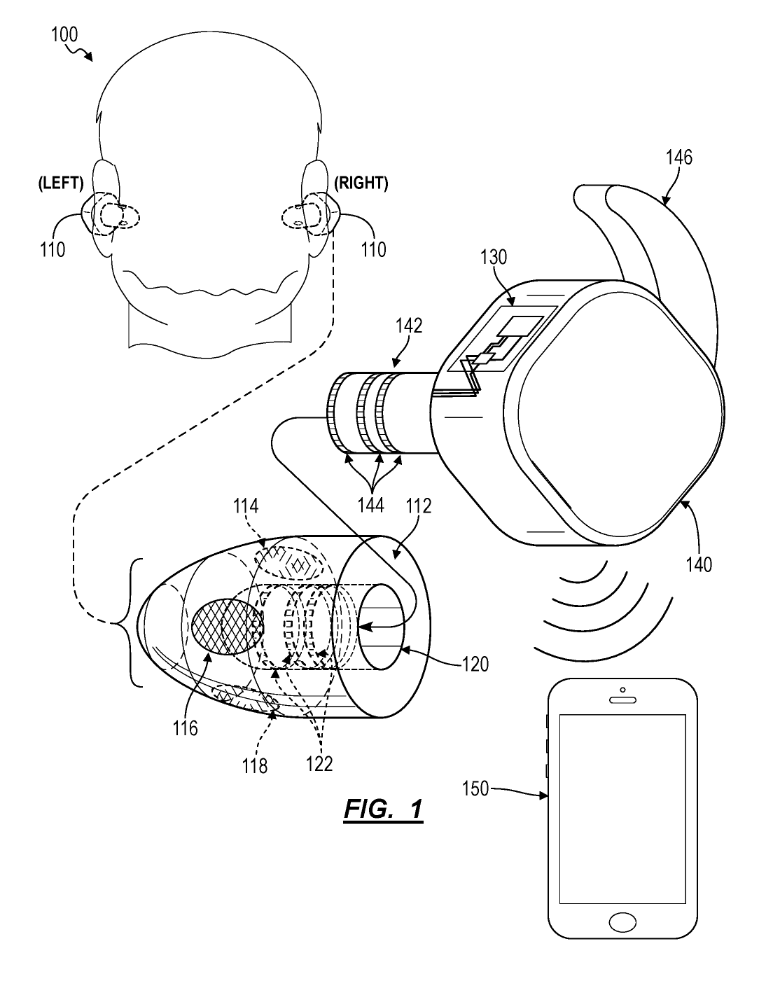

As shown in FIG. 1, an electrode tip 110 for sensing in-ear electrical signals, includes: an elastic substrate 112 defining an outer surface and an inner surface and configured to conform the outer surface against an internal surface of an ear canal of a user when the electrode tip 112 is inserted into the ear canal of the user; a sense electrode 114 arranged on the outer surface of the elastic substrate 112; a reference electrode 116 arranged on the outer surface of the elastic substrate; a driven ground electrode 118 arranged on the outer surface of the elastic substrate. The electrode tip 110 also includes an interface 120 coupled to the inner surface of the elastic substrate 112 and configured to transiently engage an earpiece housing and expose: a first electrical tab 122 electrically coupled to the sense electrode 114; a second electrical tab 122 electrically coupled to the reference electrode 116; and a third electrical tab 122 electrically coupled to the driven ground electrode 118.

As shown in FIG. 1, a system 100 for sensing in-ear electrical signals includes a left electrode tip, a right electrode tip and a signal acquisition subsystem 130. The left electrode tip includes: a left elastic substrate defining an outer surface and configured to conform the outer surface against an internal surface of a left ear canal of a user; a left sense electrode arranged on the outer surface of the left elastic substrate; a left reference electrode arranged on the outer surface of the left elastic substrate; and a left driven ground electrode arranged on the outer surface of the left elastic substrate. The right electrode tip includes: a right elastic substrate defining an outer surface and configured to conform the outer surface against an internal surface of a right ear canal of a user; a right sense electrode arranged on the outer surface of the right elastic substrate; a right reference electrode arranged on the outer surface of the right elastic substrate; and a right driven ground electrode arranged on the outer surface of the right elastic substrate. The signal acquisition subsystem 130 is configured to, during a sampling period: output a left time series of a left voltage differential between the left sense electrode and the right reference electrode; and output a right time series of a right voltage differential between the right sense electrode and the left reference electrode.

### 2. METHOD

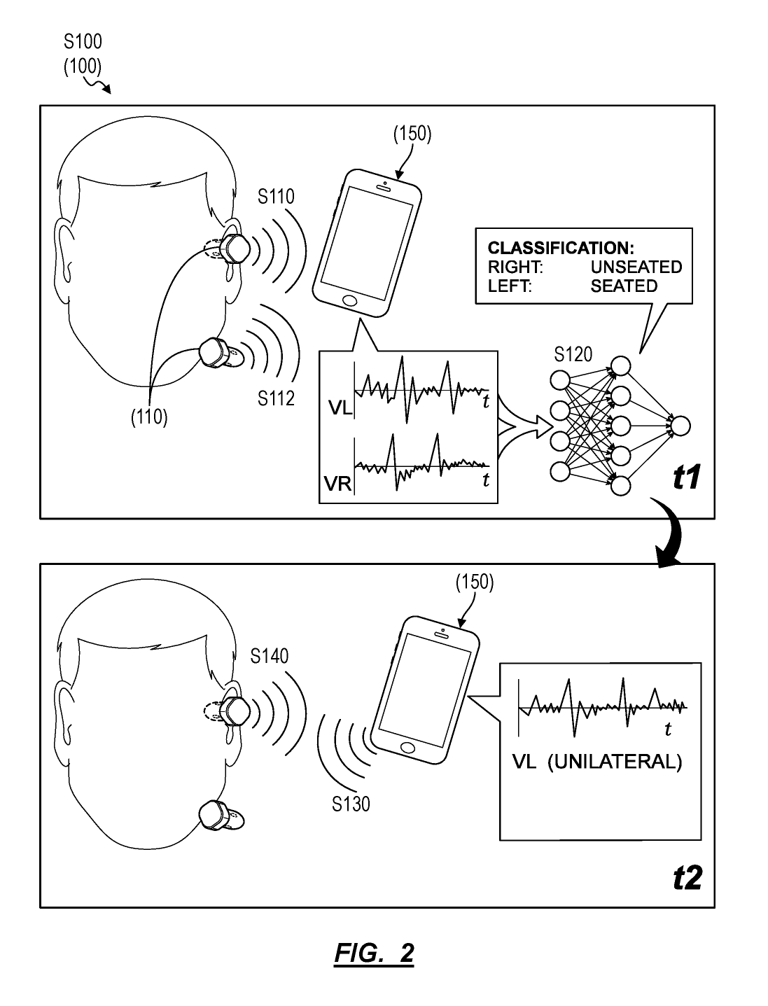

As shown in FIG. 2, a method S100 for extracting in-ear electrical signals from a user includes, during a first period: receiving, from a left electrode tip in a bilateral output mode, a first left voltage signal representing potential difference between a left sense electrode coupled to the left electrode tip, and a right reference electrode coupled to a right electrode tip in Block S110; and receiving, from the right electrode tip in a bilateral output mode, a first right voltage signal representing potential difference between a right sense electrode coupled to the right electrode tip, and a left reference electrode coupled to the left electrode tip in Block S112. The method S100 also includes detecting whether the left electrode tip is seated within an ear canal and whether the right electrode tip is seated within an ear canal based on the first left voltage signal and the first right voltage signal in Block S120. The method S100 further includes, in response to detecting that the left electrode tip is seated within an ear canal and that the right electrode tip is not seated within an ear canal during a second period: transmitting a command to a signal acquisition subsystem, electrically coupled to the left electrode tip and the right electrode tip, the command instructing the signal acquisition subsystem to switch the bilateral output mode of the left electrode tip to a unilateral output mode of the left electrode tip in Block S130; and receiving, from the left electrode tip in the unilateral output mode, a second left voltage signal representing potential difference between the left sense electrode and the left reference electrode based on the second left voltage signal and the second right voltage signal in Block S140.

### 3. APPLICATIONS

Generally, the system 100 includes a pair of electrode tips, each of which can integrate with a signal acquisition subsystem (e.g., housed in an earpiece coupled to each electrode tip), and a controller 150 to execute the method S100 in order to: detect sense and reference signals (e.g., EEG signals) from within the ear canals of a user; amplify the electrical signals; compensate for electrical interference; reject sources of noise; and classify a mental state of the user based on the sensed electrical signals. In particular, each of the electrode tips 110 includes a sense electrode, a reference electrode, and a driven ground electrode arranged on the outside surface of an elastic substrate configured to fit within the ear canal of a user. The elastic substrate 112 presses each of the electrodes against the inside surface of a user's ear canal thereby maintaining consistent electrical contact between each electrode and the skin of the user even as the user moves about and performs typical daily activities. Therefore, the system 100 can sense electrical signals from a consistent location without requiring the precise adjustment and configuration of an EEG headset. Additionally, a user can wear the system 100 for a longer period of time, thereby allowing for longer data collection periods and, therefore, better classification of a user's mental state when compared to an EEG headset.

The electrode tips 110 can also include an interface, on an internal surface of the elastic substrate 112, configured to transiently engage with the signal processing hardware and to enable electrical contact between each reference electrode and the amplifiers and analog circuitry within the signal processing hardware. Thus, the electrode tips 110 can be removed and replaced as they exhibit wear from exposure to moisture within the ear canal of a user. Additionally, the system 100 can include electrode tips of various sizes to fit a variety of ear canal sizes of a user.

Depending on the implementation, the signal acquisition subsystem 130 can be housed within two separate earpiece housings 140 or within a single housing that includes a neckband connecting each earpiece. In either case, the system 100 can include amplifiers, analog-to-digital converters (hereinafter “ADCs”), and one or more wireless transmitters in order to communicate the electrical signals sensed at the electrode tip 110 to the controller 150, which can be remote from the earpieces and electrode tips.

In an implementation of the system 100 including a single housing, the system 100 can include a split driven ground electrode that is located on both electrode tips. The system 100 can further include a driven right leg circuit connected to both sections of the split driven ground electrode in order to improve the rejection of common-mode interference.

In response to sensing electrical signals from each electrode tip, the signal acquisition subsystem 130 can switch between two different output modes. In a first mode, the signal acquisition subsystem 130 measures voltage differentials (voltage signals) between a sense electrode located on one electrode tip and a reference electrode located on the other electrode tip for each electrode tip. In a second input mode the signal acquisition subsystem 130 measures voltage differentials between a sense electrode and a reference electrode located on the same electrode tip. The system 100 can include analog amplifier circuits to amplify analog voltage differentials corresponding to either of the two input modes and an input mode switch to alternate between input modes. In one implementation, the system 100 can switch between input modes in response to the removal or insertion of an electrode tip into the ear canal of a user. For example, if a user has seated a pair of electrode tips into her ear canals, then the system 100 can operate in the first input mode (measuring voltage differentials between opposite sense and reference electrodes). Subsequently, upon detecting that the user has removed one of the electrode tips 110 from one of her ear canals, the system 100 can switch to the second input mode (measure a voltage signal between sense and reference electrodes in the same electrode tip).

In one implementation, the system 100 can include additional biometric sensors to provide additional data to the controller 150 in order to improve the noise reduction and classification algorithms. The additional biometric sensors can include a heartrate sensor, a pulse oximeter, and/or a galvanic skin response (hereinafter “GSR”) sensor. Furthermore, the system 100 can include sensors such as an accelerometer and/or gyroscope in order to detect the orientation of electrode tips. The system 100 can similarly transmit signals from the additional sensor to the controller 150 for further processing.

The controller 150 can record each of the voltage signals over a period of time to generate a time series for each measured voltage differential. The controller 150 can then filter and scale the time series and/or identify sources of noise in the time series. Subsequently, the controller 150 can generate a vector for input into one or more classification algorithms (e.g., a support vector machine, decision tree, random forest, single or multilayer neural network, k-nearest neighbor, logistic regression, Naive Bayes, linear discriminant analysis, stochastic gradient boosting, and/or Ada boosting) to classify a mental state of a user.

For ease of description, various elements of the system 100 are referred to as “a right” element or “a left” element. This terminology indicates that each element referred to as a “right” element are all associated with the same side of the system, thereby distinguishing them from those elements labeled as a “left” element. However, characteristics or functions as described with reference to “right” elements are also applicable to “left” elements.

### 4. EXAMPLES

The system 100 can be configured to enable a user to initiate a variety of actions within remote computer systems by providing a brain-computer interface that can detect specific mental states and trigger actions within a remote computer system.

In one example, the system 100 can detect the mood of the user in response to audio played via integrated in-ear headphones. The system 100 can then trigger a music player to change the characteristics of music being played according to the mood of the user, such as by playing upbeat music when the user is classified as having happy mental states, or music characterized by minor tonality when the user is classified as having a sad mental state, etc.

In another example, the system 100 can detect particular mental states of the user and trigger particular actions within a virtual environment, such as a video game or other application. For example, the system 100 can detect a motor imagery mental state and replicate the movement in an avatar representation of the user in a virtual environment, such as by moving an arm of an avatar in response to classifying a mental state as an imagined arm movement, moving a window sideways in response to classifying a thought as an imagined swiping gesture, etc.

In yet another example, the system 100 can detect particular mental states from a user and trigger connected devices to perform physical actions. For example, the system 100 can detect a particular thought and, in response, trigger a door to open, toggle a light switch, or unlock a safe, etc.

In an additional example, the system 100 can detect particular mental states of the user and provide feedback in order to aid a user in attaining a particular mental state. For example, the system 100 can detect a state of concentration of a user and, in response to a lack of concentration, play instrumental music. Alternatively, the system 100 can detect a meditative state of the user or a sleeping state of the user, and provide media to aid in meditation or sleeping.

### 5. HARDWARE

As shown in FIG. 1, the system 100 can include electrode tips 110, an interface 120 configured to transiently couple the electrode tips to an earpiece housing 140, a signal acquisition subsystem 130 housed within the earpiece housing, and a controller 150, which can interpret the electrical signals detected by the electrode tips 110 and pre-processed by the signal acquisition subsystem 130. Depending on the implementation, the system 100 can include any subset of the above components. For example, the system 100 can include a single electrode tip, a pair of electrode tips, a pair of electrode tips and the signal acquisition subsystem 130 and associated housing, or all of the above-mentioned components. In implementations wherein, the system 100 includes less than all of the above-mentioned components, the system 100 can be configured to interface with third-party devices in order to perform steps of the method S100. In one implementation, the system 100 includes a left earpiece transiently coupled to the left electrode tip, wherein the left earpiece is seated within the left ear canal of the user and configured to occupy a left outer ear of the user when the left electrode tip; and a right earpiece is seated within the right ear canal of the user and transiently coupled to the right electrode tip, wherein the right earpiece configured to occupy a right outer ear of the user when the right electrode tip.

In particular, the system 100 is configured to be worn by a user in a similar manner to a set of in-ear headphones, though in some implementations the system 100 does not perform audio playback functions. For example, the system 100 can include two electrode tips, each transiently engaged with a separate wireless housing, which can communicate with a remote controller 150. In this example, each wireless housing can be supported proximal to the outer ear of the user by externally directed pressure created by the electrode tip 110 pressing against the inside of the user's ear canal. In another example, the two separate housings are connected via a neckband or other physical bridge, which can communicate wirelessly with a remote controller 150 via a single transceiver. The remote controller 150 can include a standalone device configured to execute the method S100, which can be configured with the form factor of a wearable device such as a watch. Alternatively, the functions of the controller 150 can be performed by executing an application on a portable computational device such as a smartwatch, smartphone, tablet computer, laptop computer, etc. In an alternative implementation, the controller 150 is not remote to the earpiece housing 140 of the system 100 and instead is located within the housing (e.g., within a housing located on a neckband or within an earpiece housing 140 on one or both ears).

The system 100 can also include additional components, such as a gyroscope and/or accelerometer in order to detect the orientation of each earpiece and to detect potential sources of noise (e.g., movements of the jaw or head) in order to characterize the status of the earpieces (e.g., whether each of the earpieces are secured in the user's ears).

### 5.1 Electrode Tips

As shown in FIG. 1, an electrode tip of the system 100 can include: an elastic substrate defining an outer surface and an inner surface and configured to conform the outer surface against an internal surface of an ear canal of a user when the electrode tip 110 is inserted into the ear canal of the user; a sense electrode arranged on the outer surface of the elastic substrate 112; a reference electrode arranged on the outer surface of the elastic substrate 112; a driven ground electrode arranged on the outer surface of the elastic substrate 112; and an interface coupled to the inner surface of the elastic substrate 112 and configured to transiently engage an earpiece housing 140 and expose: a first electrical tab 122 electrically coupled to the sense electrode 114; a second electrical tab 122 electrically coupled to the reference electrode 116; and a third electrical tab 122 electrically coupled to the reference electrode 116.

In particular, the system 100 can include the sense electrode 114, the reference electrode 116, and the driven ground electrode 118 radially arranged (e.g., radially offset from each other) on the outer surface of the elastic substrate 112. In this configuration, the system 100 can ensure electrical isolation between the electrodes and improve the signal-to-noise ratio (hereinafter “SNR”) of the voltage differential measured between the sense electrode 114 and the reference electrode 116 on the same electrode tip. Additionally or alternatively, the system 100 includes the sense, reference, and driven ground electrodes laterally offset along the length of the elastic substrate of the electrode tip 110.

In one implementation, the system 100 is configured with the driven ground electrode 118 arranged downward on the outer surface of the elastic substrate 112 when the electrode tip 110 is inserted into the ear canal of the user. The sense electrode 114 and the reference electrode 116 can then be arranged radially offset from the driven ground electrode 118 on the outer surface of the elastic substrate 112. In one implementation, the sense electrode 114 is arranged such that it faces toward the temporal and/or frontal lobe of a user's brain when the electrode tip is inserted within the ear canal of a user while the reference electrode 116 is arranged toward the occipital lobe of the user's brain when the electrode tip 110 is inserted into an ear canal of a user.

In an alternative implementation as shown in FIG. 4, the system 100 is configured with the driven ground electrode 118 arranged facing downward and forward on the outer surface of the elastic substrate 112 when the electrode tip 110 is inserted into the ear canal of the user. The sense electrode 114 is arranged facing upward on the outer surface of the elastic substrate 112 when the electrode tip 110 is inserted into the ear canal of the user. The reference electrode 116 is arranged facing downward and backward on the outer surface of the elastic substrate 112.

The system 100 can also include electrode tips of multiple sizes, each size configured to fit a different range of ear canal sizes. Furthermore, the system 100 can include electrode tips configured to engage various housing configurations or earpiece form factors.

### 5.1.1 Elastic Substrate

Generally, the elastic substrate 112 defines an outer surface and an inner surface and is configured to conform its outer surface against an internal surface of an ear canal of a user when the electrode tip 110 is inserted into the ear canal of the user. The elastic substrate 112 can be constructed from a medium density foam, silicon, or elastic polymer material. The system 100 includes a medium density elastic substrate such that the elastic substrate is dense enough to exert an outward pressure on the inner surface of a user's ear canal in order to maintain electrical contact between the electrodes and the user's inner ear and to support an earpiece transiently engaged with the electrode tip 110. However, the material of the elastic substrate 112 can have a low enough density that the elastic substrate 112 can be compressed before insertion into the ear canal of a user (e.g., such that the elastic substrate 112 can later expand and anchor itself within the ear canal of the user) and such that the elastic substrate 112 does not exert so much pressure on the ear canal of the user as to cause discomfort or irritation.

As shown in FIG. 4, the elastic substrate 112 can include three lobes, each lobe arranged in alignment with one of the electrodes such that: a first lobe is configured to project outward the outer surface of the elastic substrate 112 at a location of the sense electrode 114 on the outer surface of the elastic substrate 112; a second lobe is configured to project outward the outer surface of the elastic substrate 112 at a location of the reference electrode 116 on the outer surface of the elastic substrate 112; and a third lobe is configured to project outward the outer surface of the elastic substrate 112 at a location of the driven ground electrode 118 on the surface. Implementations of the system 100 that include these lobes can exhibit improved performance over repeated compression of the elastic substrate 112 during insertion into an ear canal of a user by providing a trough between each lobe into which each electrode tip can be compressed. Thus, an electrode arranged over a lobe in the elastic substrate 112 does not deform (e.g., change concavity) upon being compressed.

The elastic substrate 112 can be constructed via injection molding or any other molding process and/or additive manufacturing techniques. However, the elastic substrate 112 can be constructed according to any other manufacturing technique

### 5.1.2 Electrode Construction

Generally, the sense electrode 114, the reference electrode 116, and the driven ground electrode 118 can be constructed with the same techniques and materials in order to maintain a similar level of resistance at each electrode's interface with the skin of the user within the ear canal of the user. In one implementation, each electrode includes a solid conductive metal electrode imbedded within the elastic substrate 112 such that a portion of the surface of the electrode is exposed over the surface of the elastic substrate 112. In another implementation, each electrode includes a conductive metal fabric (e.g., silver fabric) adhered or otherwise attached to the surface of the elastic substrate 112. The metal fabric (e.g., a woven metal fabric) can be configured to exhibit a level of elasticity such that each electrode can conform to the inner surface of a user's ear canal. In yet another implementation, each electrode includes a layer of conductive substrate (e.g., a silver or silver chloride ink) applied directly onto the surface of the elastic substrate 112 such that the elasticity of the electrode surface substantially matches the elasticity of the elastic substrate 112 (e.g., via printing or thin film deposition).

An electrode tip can include a sense electrode, a reference electrode, and a driven ground electrode wherein each electrode has an equal surface area such that each electrode exhibits a similar resistance at the interface 120 of the electrode and the skin of the user.

Each electrode in the electrode tip 110 can also include a corresponding electrical trace that transmits an electrical signal from the electrode to an electrical tab 122 at the interface 120 between the electrical tip and the earpiece housing 140. The electrode tip 110 can include an electrical trace in the form of an insulated wire electrically coupled to each electrode and imbedded within the elastic substrate 112.

However, the sense electrode 114, the reference electrode 116, and the driven ground electrode 118 can be constructed in any other way that results in a smooth surface facing outward from the elastic substrate 112 that makes consistent contact with the inner surface of a user's ear canal when the electrode tip 110 is inserted into the ear canal of the user.

### 5.1.3 Sense and Reference Electrode

Each electrode tip 110 includes a sense electrode 114 and reference electrode 116 pair that establishes a voltage signal measured as a potential difference between the sense electrode 114 and the reference electrode 116. The sense electrode 114 and the reference electrode 116 for which the system 100 measures a voltage signal can be located on the same electrode tip 110 or on separate electrode tips 110 in each ear canal of a user. For example, the system 100 can measure a voltage differential between a sense electrode located on a left electrode tip 110 and a reference electrode located on right electrode tip 110 and vice versa. In an alternative example, the system can measure a voltage differential between a sense electrode on a left electrode tip 110 and a reference electrode on the same electrode tip 110.

In one implementation, the reference electrode 116 is arranged on the earpiece housing 140 proximal the concha of the user when the electrode tip 110 is inserted into the ear canal of the user instead of being located on the electrode tip 110 itself.

Generally, each electrode tip 110 includes a single sense electrode 114 and reference electrode pair. However, in some implementations, an electrode tip 110 can include additional sense electrode and reference electrode pairs positioned on the surface of the electrode tip 110. Alternatively, the system 100 can include multiple sense electrodes 114 with only one reference electrode 116. In one implementation, the system 100 includes electrode tips including only the sense electrode 114 and measures a voltage differential between the sense electrode 114 and the common circuit voltage of the signal acquisition subsystem 130.

### 5.1.4 Additional Biometric Sensors

The system 100 can also include additional biometric sensors including a heartrate sensor and a galvanic skin response sensor. In one implementation, each electrode tip 110 includes a heart rate electrode in addition to the sense electrode 114, the reference electrode 116, and the driven ground electrode 118. In this implementation, the heartrate electrode can be located proximal to a major blood vessel (e.g., artery or vein) close to the inner surface of the ear canal when the electrode tip 110 is seated in the ear canal of the user. For example, the heartrate electrode can be located on the surface of the elastic substrate 112 closest to the superficial temporal blood vessels. The heartrate electrode can then transmit a heartbeat signal to be interpreted at the controller 150 to determine a user's heartrate or heartrate variability during a sampling period. Additionally or alternatively, the system 100 can include a pulse oximeter located proximal to the superficial temporal blood vessels, or elsewhere in the ear canal, in order to detect blood oxygen levels of the user.

The system 100 can further include a GSR electrode, which can measure the skin conductance of the skin in a user's ear canal.

### 5.1.5 Driven Ground Electrode

Each electrode tip 110 can also include a driven ground electrode 118 that functions to reduce the common mode signal present at the sense electrodes 114 and reference electrodes 116. In one implementation, the driven ground electrode 118 is connected to a driven right leg circuit in order to reduce common-mode interference at the sense electrode 114 and the reference electrode 116. In implementations wherein, the reference electrode 116 is located at the concha of a user, the system 100 can function without a driven ground electrode 118.

### 5.1.6 Split Driven Ground Electrodes

In one implementation, the system 100 includes a split driven ground electrode wherein the left driven ground electrode is electrically coupled to the right driven ground electrode to form a driven right leg electrode 119. In this implementation, each side of the split driven ground electrode can be characterized by a surface area that is half of the surface area of either the sense electrodes 114 or the reference electrodes 116 such that the total surface area of the split driven ground electrode is equal to the surface area of each sense electrode 114 or reference electrode 116.

Generally, the split driven ground electrode provides better common-mode interference rejection and improves the signal to noise ratio of the differential voltages measured between the sense electrodes 114 and the reference electrodes 116. By including a split driven ground electrode with a total surface area equal to the surface area of each of the sense electrodes 114 and reference electrodes 116, the system 100 ensures that the input impedance at each electrode is as close to equal as possible in order to reduce common mode interference between the sense electrode 114 and the reference electrode 116.

### 5.1.7 Internal Interface

Each electrode tip 110 can include an interface 120 coupled to the inner surface of the elastic substrate 112 and configured to transiently engage an earpiece housing 140 and expose: a first electrical tab 122 electrically coupled to the sense electrode 114; a second electrical tab 122 electrically coupled to the reference electrode 116; and a third electrical tab 122 electrically coupled to the reference electrode 116. Generally, the electrode tip 110 can include a cylindrical or conical inner surface onto which the electrical tabs 122 can be arranged. The inner surface of the elastic substrate 112 can be configured to transiently engage with a conductive protrusion 142 of the earpiece housing 140. The conductive protrusion 142 can include corresponding conductive tabs that contact the electrical tabs 122 on the inner surface of the elastic substrate 112.

In one implementation, the interface 120 includes a set of laterally offset concentric conductive rings, each conductive ring electrically coupled to one of the electrodes (via an insulated wire imbedded in the elastic substrate 112) and lining the internal surface of the elastic substrate 112. The conductive protrusion 142 can include corresponding concentric rings on its outside surface to engage with the concentric rings on the internal surface of the elastic substrate 112. Thus, the electrical signals detected at the electrodes can propagate through the electrode tip 110 to the signal processing hardware in the earpiece housing 140 via the internal interface of the electrode tip 110.

However, the system can include any other attachment means between the electrode tips 110 and the earpiece housing 140 that maintains electrical contact between the electrodes on each electrode tip 110 and the signal acquisitions subsystem 130.

### 6. SIGNAL ACQUISITIONS SUBSYSTEM

Generally, the signal acquisition subsystem 130 can include signal processing hardware configured to amplify, denoise, digitalize, and transmit and/or output the analog electrical signals detected at the sense electrode 114 and the reference electrode 116. In particular the system 100 can include operational amplifiers, an ADC, and a transceiver. The system 100 can also include a driven right leg circuit for each electrode tip 110 or a single driven right leg circuit for both electrode tips 110. Thus, via the signal acquisition subsystem 130, the system 100 can: sense a left voltage differential between a left sense electrode 114, coupled to the left electrode tip 100, and a right reference electrode 116, coupled to the right electrode tip 100; and sense a right voltage differential between a right sense electrode 114, coupled to the right electrode tip 110, and a left reference electrode 116, coupled to the left electrode tip 110.

### 6.1 Amplifiers and Filters

The system 100 can include high input impedance instrumentation amplifiers to amplify the voltage differential at each electrode channel from the common-mode signal to generate a differential signal. In one implementation, the system 100 can include an analog lowpass filter at 0.5 Hz to remove low frequency artifacts (e.g., a heartbeat rhythm) from the amplified differential signal extracted from each sense electrode 114 and reference electrode 116.

### 6.2 Driven Right Leg Circuit

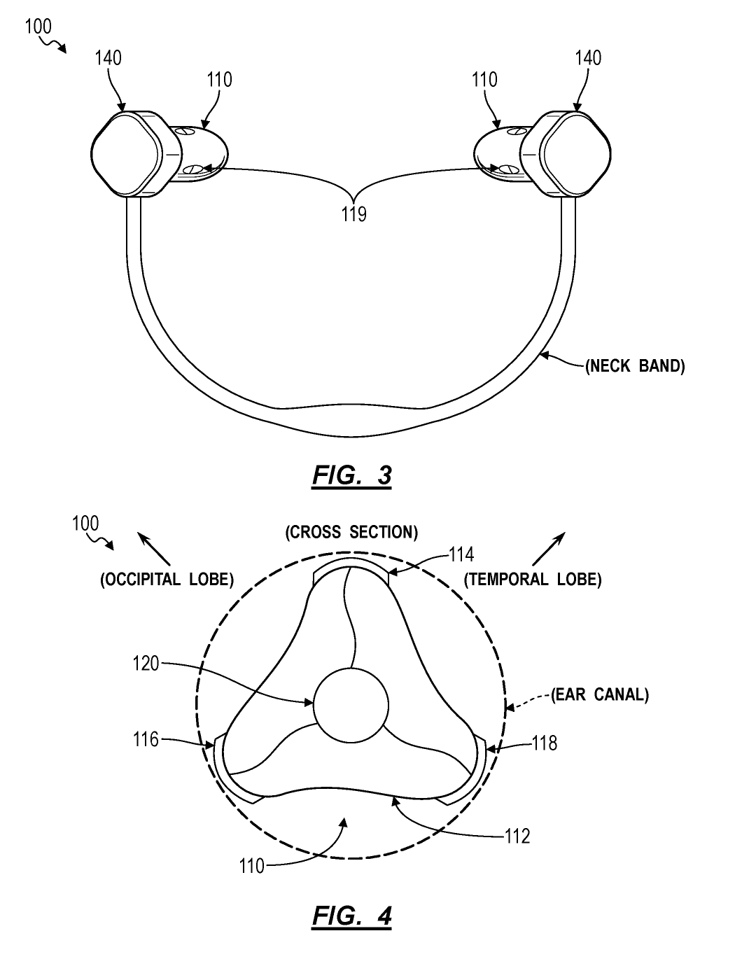

In one implementation, as shown in FIG. 3 the system 100 includes: a driven right leg circuit electrically coupled to a driven right leg electrode (or driven ground electrode) and configured to reduce common-mode interference in the left voltage differential and the right voltage differential. The method S100 can also include, during the first sampling period: canceling common-mode interference in the left voltage differential and the right voltage differential via a driven right leg circuit, wherein the driven right leg circuit is electrically coupled to a split driven ground electrode seated in both ear canals of the user.

In particular, the driven right leg circuit functions to reduce the common-mode signal present at the sense electrode 114 and the reference electrode 116 (e.g., by driving current 180 degrees out of phase with the common-mode signal to the sense electrodes 114 and the reference electrodes 116).

### 6.3 Analog-to-Digital Converter

The system 100 can include an ADC configured to sample the analog differential signal from each sense electrode 114 and reference electrode 116. In one implementation, the system 100 includes a sigma-delta ADC with 24-bits of resolution. In an alternative implementation, the system 100 includes a successive approximation ADC with 24-bits of resolution.

However, the system 100 can include any other type of ADC depending on the implementation.

### 6.4 Transceiver

The system 100 can also include a transceiver to send and receive the digitalized signals to the controller 150 (in implementations wherein the controller is remote to the earpiece housing 140) for further processing and mental state classification. After the voltage differential (e.g., the voltage signal) from each electrode channel has been digitalized via the ADC, the system 100 can transmit the digital samples of the voltage differential to the controller 150 for further processing and mental state classification. In some implementations, the transceiver can also receive signals from the controller 150 or another computational device in order to enable functions of the system 100 such as changing the input mode of the system 100 or generating an audio signal via integrated in-ear headphones.

### 6.5 Analog Input Switch

In one implementation, the signal acquisition subsystem 130 is configured to respond to commands received from the controller 150 in order to activate an analog input switch, which changes the input mode of the differentially amplified voltages. For example, the signal acquisition subsystem 130 can initially be configured to output a left voltage signal (by amplifying a differential voltage between the left sense electrode and the right reference electrode) and a right voltage signal (by amplifying a differential voltage between the right sense electrode and the left reference electrode) in a bilateral input mode. However, in response to receiving a command from the controller (e.g., due to detecting that one of the electrode tips is not seated in an ear canal), the signal acquisition subsystem 130 can activate a switch, which changes the input mode to a unilateral input mode. In the unilateral input mode, the signal acquisition subsystem 130 outputs a left voltage signal by amplifying the differential voltage between the left sense electrode 114 and the left reference electrode 116 and outputs a right voltage signal by amplifying the differential voltage between the right sense electrode 114 and the right reference electrode 116.

### 7. EARPIECE HOUSING

Generally, the system 100 includes earpiece housings 140 (i.e. earpieces) that enclose the signal acquisitions subsystem and engage with the electrode tips 110 via a conductive protrusion 142 (or any other attachment mechanism) from each earpiece housing 140 and the internal interface of each electrode tip 110. The system 100 can include various housing configurations for the earpiece housings 140. In one implementation, the system 100 includes two separate earpiece housings 140 in the form of wireless earpieces. Alternatively, the system 100 includes two earpiece housings 140 connected by a neckband to be worn around the back of the neck of the user. In some implementations, the earpiece housing 140 encloses additional components such as an accelerometer, audio, processor and/or a headphone audio system within the earpiece housings 140.

In another implementation, the left earpiece includes a left physical reference 146 configured to rest against a left concha of a left ear of the user when the left earpiece is worn by the user; and the right earpiece includes a right physical reference 146 configured to rest against a right concha of a right ear of the user when the right earpiece is worn by the user. Thus, the system 100 can include a physical reference 146 that abuts anatomical features of the outer-ear of the user in order to consistently locate and orientate each earpiece housing 140 and therefore each electrode tip 110 within the ear canal of a user. In one implementation, the physical reference 146 comprises a soft rubber, silicon, or elastic polymer extrusion from the earpiece housing 140 that is configured to rest against the concha of a user's ear. Alternatively, the system 100 can include physical reference 146 structures that wrap around the ear or otherwise fix the earpiece housing 140 relative to the ear canal of the user, thereby rotationally and/or laterally constraining the electrode tip 110 installed on the earpiece housing 140 within the ear canal of the user.

Additionally, the earpiece housing 140 can include a conductive protrusion 142 configured to engaged with the interface 120 of an electrode tip 110. Just as the interface 120 includes electrical tabs 122 electrically coupled to each sense electrode 114, reference electrode 116, and driven ground electrode 118, the conductive protrusion 142 includes electrical contact regions 144 configured to conduct signals from the electrical tabs 122 in the interface 120 to the signal acquisition subsystem 130. For example, if the interface 120 of an electrode tip 110 includes three electrical tabs 122 (e.g., for the sense electrode, reference electrode, and driven ground electrode), then the conductive protrusion 142 includes three corresponding electrical contact regions 144 arranged such that, when the earpiece housing 140 is engaged with the electrode tip 110, each of the electrical contact regions 144 align with a corresponding electrical tab 122.

### 7.1 Neckband Configuration

The system 100 can also include a neckband connecting each of the earpiece housings 140 in order to electrically couple various components between the two earpiece housings 140 and/or electrode tips 110. For example, the split driven ground electrode or the split driven ground electrode includes an electrical connection between the driven ground electrode in each electrode tip 110 which requires a physical wire to connect each earpiece housing. The neckband therefore provides a housing for this wire. Additionally, the neckband configuration can enable other implementations, such as including analog amplification of a differential voltage between a sense electrode 114 in one electrode tip 110 and a reference electrode 116 in another electrode tip 110. For example, an amplifier can be electrically coupled to both a sense electrode 114 in the left electrode tip 110 and a reference electrode 116 in a right electrode tip 110.

In this implementation, the transceiver and other signal processing components can also be housed within a separate housing located within the neckband or within either of the earpiece housings 140. Additionally, in implementations including the neckband, the system 100 can include a single battery to power the system 100.

### 7.2 Wireless Configuration

The system 100 can also include a wireless earpiece configuration wherein each earpiece housing 140 is a separate earpiece. In this implementation, each earpiece housing 140 encloses a transceiver to separately transmit the digitalized voltage differentials measured at the electrodes to the controller 150. As such, each separate earpiece housing 140 also includes an ADC, amplifiers, and driven ground circuitry in order to extract the differential signals from the sense electrodes 114 and reference electrodes 116. Additionally, each earpiece housing 140 can also include its own battery to power operation of each separate wireless earpiece. In implementations of the system 100 including two wireless earpieces, as opposed to two earpieces connected by a neckband, the system 100 can exhibit improved usability and a reduced likelihood of tangling or potential irritation of the neck band on the back of the user's neck.

In one implementation, the system 100 includes integrated wireless headphones, which can also include an audio speaker and other audio components to enable various audio playback functions. Thus, the left electrode tip 110 includes a left interface coupled to an inner surface of the left elastic substrate and configured to transiently engage a left earpiece; the right electrode tip 110 includes a right interface coupled to an inner surface of the right elastic substrate and configured to transiently engage a right earpiece; and the controller 150 is remote from the left earpiece and the right earpiece. Therefore, the system 100 includes the left earpiece configured to transmit the left time series to the controller 150; and the right earpiece configured to transmit the right time series to the controller 150.

### 8. CONTROLLER

The system 100 includes a controller 150 that is configured to, during a sampling period: record a left time series of a left voltage differential between the left sense electrode 114 and a first reference electrode 116 in a set of reference electrodes 116 comprising the left reference electrode 116 and the right reference electrode 116; and record a right time series of a right voltage differential between the right sense electrode 114 and a second reference electrode 116 in the set of reference electrodes 116. The controller 150 is also configured to, based on the left time series and the right time series, classify a mental state of the user during the sampling period.

### 9. DIGITAL SIGNAL PROCESSING

Once the electrical signals from each electrode in the electrode tip 110 have been digitalized at the ADC and transmitted or otherwise communicated to the controller 150, the system 100 (e.g. at the controller 150) can execute digital signal processing techniques to: receive digitalized voltage signals from the earpieces and installed electrode tips 110 in Blocks S110, S112, S140; detect whether each electrode tip 110 is seated within an ear canal in Block S120; change the input mode between a bilateral input mode and a unilateral input mode in response to detecting that one of the electrode tips 110 is not seated within an ear canal in Blocks S130; reduce and/or reject noise in the voltage signals; filter and scale the voltage signals; generate an input vector to various classification models; and classify a mental state of a user based on the input vector.

### 9.1 Input Modes

In Block S130, the system 100 can extract a left voltage signal and right voltage signal from the left voltage signal and the right voltage signal according to a particular input mode. In implementations wherein the left earpiece and the right earpiece are physically connected, the system 100 can operate in a bilateral or unilateral configuration. In a unilateral configuration, the system 100 can record a voltage signal from each earpiece individually, thereby enabling the user to wear a single earpiece and still record a voltage signal from that earbud that can be classified as a particular mental state. In the bilateral configuration, the system 100 records voltage signals of voltage differentials measured between opposite earpieces worn by the user, which can improve the SNR of the differential signal.

In one example, in a bilateral configuration, the system 100 can record a left voltage signal based on the voltage differential between the left sense electrode 114 and the right reference electrode 116 and a right voltage signal based on the voltage differential between the right sense electrode 114 and the left reference electrode 116. Alternatively, in a unilateral configuration, the system 100 can record a left voltage signal between the left sense electrode 114 and the left reference electrode 116 and a right voltage signal between the right sense electrode 114 and the right reference electrode 116.

Additionally, the system 100 can implement the unilateral configuration with only one earpiece (or switch from a bilateral configuration to a unilateral configuration) by: detecting a seated electrode tip 110 in the pair of electrode tips 110 and an unseated electrode tip 110 in the pair of electrode tips 110, wherein the seated electrode tip 110 is seated within an ear canal of the user and the unseated electrode tip 110 is not seated within an ear canal of the user; in response to detecting the seated electrode tip 110 and the unseated electrode tip 110, recording a second voltage signal of a voltage differential between a seated sense electrode 114 of the seated electrode tip 110 and a seated reference electrode 116 of the seated electrode tip 110; and based on the second voltage signal, classifying a mental state of the user during the second sampling period.

The system 100 can detect whether each electrode tip 110 is seated within the ear canal of the user by classifying the voltage signal recorded during the first sampling period as being seated or not seated. Additionally, the system 100 can detect the orientation of the electrode tips 110 relative to each other (e.g., via the accelerometer and/or gyroscope included in each earpiece) and relative to the force of gravity in order to virtually position each earpiece in three-dimensional space. The system 100 can then establish a threshold relative position for each earpiece relative to each other and indicate that at least one electrode tip 110 is not seated with an ear canal of the user if the position of the earpieces is outside of the threshold.

In one implementation, the system 100 executes an insertion classifier to detect whether at least one of the electrode tips 110 are not seated within an ear canal of a user. The insertion classifier can include any of the classification techniques described below in order to classify each electrode tip 110 as either seated or unseated with respect to a user's ear canal. The system 100 can execute an insertion classifier that takes as input accelerometer and gyroscopic data that were recorded during a relevant sampling period.

### 9.2 Noise Reduction

The system 100 can identify and remove sources of noise from the time series data collected from each electrode tip 110 in order to better classify a mental state of the user. The system 100 can include additional sensors within each earpiece, such as an accelerometer, gyroscope, microphone, or any other sensors that can detect sources of noise in the environment.

In one implementation, the system 100 records a time series of acceleration and gyroscopic data within the same sampling period during which time series of voltage differential data are recorded in order to determine intervals within the sampling period during which significant movement has occurred. After identifying intervals of movement based on the accelerometer and gyroscopic data, the system 100 can remove corresponding intervals (e.g., recorded at the same time) of the time series of voltage differential data in order to remove data that may be potentially corrupted by motion artifacts.

Additionally or alternatively, the system 100 can detect motion artifacts directly from the time series of voltage differential data by classifying subsections of each sampling period of the series of voltage differential data according to known motion artifacts. For example, the system 100 can measure a signal pattern that may be characteristic of the user masticating and can characterize this pattern according to a machine learning algorithm (e.g., a convolutional neural net, long short-term memory recurrent neural network, etc.). Upon detecting a motion artifact attributable to a known source (e.g., masticating) the system 100 can remove an interval of the time series of voltage differential data corresponding to the detected artifact.

In one implementation, the system 100 includes a microphone that can: measure a series of audio samples; detect, from the audio samples, audio signals generated by the user (e.g., by talking or chewing); and correlate the audio signals with motion artifacts. Thus, the system 100 can detect sounds that can be correlated with the appearance of motion artifacts in the voltage differential signals. For example, the system 100 can detect sounds caused by the user masticating or speaking. The system 100 can then measure the interval of these sounds and remove voltage differential data corresponding to the measured interval.

### 9.2 Filtering and Scaling

After removing sources of noise, the system 100 can digitally filter and scale the voltage differential signals in order to improve classification of the voltage differential signals. In some implementations, the controller 150 applies bandpass, highpass, and lowpass filters to remove noisy or irrelevant frequency components from the voltage differential signals. The system 100 can also calculate the mean each voltage signal and can remove the mean in order to calculate a variance signal from the voltage differential signal. The variance signal may improve classification by better representing EEG signals from the brain of the user.

In one implementation, the system 100 applies a digital bandpass filter (e.g., a seventh-order bandpass filter) between 0.5 Hz and 50 Hz in order to remove 60 Hz noise from the signal. Additionally or alternatively, the system 100 can calculate the mean of each voltage signal over a sampling interval (e.g., 0.5 seconds) and can subtract the calculated mean from the voltage signal. Furthermore, the system 100 can scale the voltage different signals by the variance of the signal in order to normalize the signal between users and between sessions of the same user.

In one implementation, the system 100 includes greater than two voltage differential signals and executes a spatial filter to maximize the variance between multivariate signals. For example, the system 100 can execute the common spatial pattern procedure to maximize the variance ratio between the voltage differential signals.

### 9.4 Input Vector

Once the system 100 has filtered and scaled the digital voltage differential signals, the system 100 can generate an input vector for the classification algorithm. The system 100 can calculate various features of the input vector based on the digital voltage differential signals, such as the mean, variance, maximum, Hjorth fractal dimension, Hurst exponent, Hjorth mobility, Hjorth complexity, multiscale entropy, Petrosian fractal dimension, spectral entropy, and the Katz fractal dimension.

Additionally or alternatively, the system 100 can calculate frequency components of the signal and input the power of each frequency component as a feature in the input vector. For example, the system 100 can calculate the absolute power of each time series of voltage differential data and then calculate the power of frequency bands, such as between 0.5 and 4 Hz, 5 and 8 Hz, 9 and 13 Hz, 14 and 18 Hz, 19-30 Hz, and 30-40 Hz. The system 100 can then scale the frequency band power by the absolute power and include the scaled power of each frequency band within the input vector for the classification algorithm.

Additionally or alternatively, the input vector can contain time-frequency domain features. The system 100 can apply a wavelet transformation to the time series of voltage differential data to obtain a two-dimensional dataset containing the time-frequency information of the voltage differential signal. The system 100 can then scale the time-frequency information by dividing each sample by the absolute signal power as discussed above.

In another implementation, the system 100 inputs the time-series of the voltage differential data directly into the classification algorithm as a set of features. For example, if the system 100 samples the sense electrodes 114 and reference electrodes 116 at 500 Hz, then the input vector for one second of data would be of length 1000 including both the left and right time series.

### 9.5 Classification

The system 100 can, based on the first left time series and the first right time series, classify a mental state of the user during the sampling period. Once the system 100 has generated an input vector, the system 100 can execute a classification algorithm on the input vector to categorize a mental state of the user during the sampling period. A mental state of a user can be a particular thought (e.g., motor imagery of particular arm movement) or simply a particular mental state (e.g., a level of concentration or emotional sentiment). The system 100 can execute one or more classification algorithms such as a support vector machine, decision tree, random forest, single or multilayer network, k nearest neighbor, logistic regression, naïve Bayes, linear discriminant analysis, stochastic gradient boosting, and/or Ada boosting. The system 100 can also implement deep learning algorithms such as artificial neural networks, deep belief networks, recurrent neural networks, long short-term memory or gated recurrent units, capsule networks, and/or generative adversarial networks. Furthermore, the system 100 can apply multiple classifiers to the left time series and the right time series of the voltage differential data. The system 100 can then output the consensus of the multiple classifiers as the final classification of a mental state of a user.

In one implementation, the left time series and the right time series are each filtered into bands between 0.5 Hz and 50 Hz. Subsequently, the system 100 utilizes a one-dimensional convolutional neural network. After three convolutional layers and three pooling layers, the system 100 can flatten the output using a fully connected five-layer artificial neural network. The system 100 can also utilize batch normalization and dropout techniques in order to prevent overfitting of the voltage differential data.

In another implementation, the classification algorithm includes a combination of convolutional and recurrent neural networks. For example, the system 100 can execute a two-dimensional convolutional neural network combined with a long short-term memory recurrent neural network in order to account for time dependent aspects of the input data.

In yet another implementation, the classification algorithm includes a generative adversarial network. The generative network of the generative adversarial network generates voltage differential data that mimics examples of real voltage differential data in order to reduce the amount of data required to train the model.

In implementations that include both unilateral and bilateral input modes, the system 100 can execute classifiers for each input mode. For example, the system 100 can execute a bilateral classifier that takes as input an input vector derived from both a left time series of voltage differential data and a right time series of voltage differential data; and a unilateral classifier that takes in a single input vector derived from a seated time series of voltage differential data.

### 9.6 False Positive Reduction

The system 100 can execute a number of techniques in order to reduce the rate of false positive classification by the system 100. In one implementation, the system 100 utilizes a shorter sampling period over which to classify mental states of the user. In this implementation, the system 100 outputs a classification only after classifying a consistent mental state across a predefined number of sampling periods. In another implementation, the system 100 can adjust a cost function of the classifier in order to bias the classifier to favor false negatives over false positives. For example, the system 100 can implement a cost function that requires the classifier to achieve 70% certainty for classifying a mental state, as opposed to a 50% certainty, which is typical.

In one implementation, the system 100 utilizes multiple different classifiers and only classifies a mental state based on a consensus classification by the multiple different classifiers. For example, the system 100 can implement a support vector machine classifier, a random forest classifier, a naïve Bayes classifier, a neural network classifier, and a k-nearest neighbors classifier, all of which are trained on the same training data. Upon receiving voltage differential data over a sampling period, the system 100 can then evaluation each of the classifiers; and, in response to a majority of the classifiers outputting the same classification, outputting the classification.

In another implementation, the system 100 can train separate classifiers for each sense electrode 114 and reference electrode 116 pair in the system 100. For example, the system 100 can train a classifier for the voltage differential signals of the left sense electrode 114 and the right reference electrode 116, as well as the voltage differential signals of the right sense electrode 114 and the left reference electrode 116. The system 100 can then output a classification in response to agreement of both classifiers. If the system 100 includes additional sense electrode 114 and reference electrode 116 pairs, the system 100 can output a classification in response to a consensus of the classifiers.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A system for sensing in-ear electrical signals, comprising:
a left electrode tip comprising:
a left elastic substrate defining an outer surface and configured to conform the outer surface against an internal surface of a left ear canal of a user;
a left sense electrode arranged on the outer surface of the left elastic substrate;
a left reference electrode arranged on the outer surface of the left elastic substrate; and
a left driven ground electrode arranged on the outer surface of the left elastic substrate;
a right electrode tip comprising:
a right elastic substrate defining an outer surface and configured to conform the outer surface against an internal surface of a right ear canal of a user;
a right sense electrode arranged on the outer surface of the right elastic substrate;
a right reference electrode arranged on the outer surface of the right elastic substrate; and
a right driven ground electrode arranged on the outer surface of the right elastic substrate; and
a signal acquisition subsystem configured to, during a first period:
output a left voltage signal representing potential difference between the left sense electrode and the right reference electrode; and
output a right voltage signal representing potential difference between the right sense electrode and the left reference electrode.

2. The system of claim 1, further comprising:
a left earpiece transiently coupled to the left electrode tip, the left earpiece configured to occupy a left outer ear of the user when the left electrode tip is seated within the left ear canal of the user; and
a right earpiece transiently coupled to the right electrode tip, the right earpiece configured to occupy a right outer ear of the user when the right electrode tip is seated within the right ear canal of the user.

3. The system of claim 2, wherein:
the left earpiece comprises a left physical reference configured to rotationally constrain the left electrode tip within the ear canal of the user when the left earpiece is worn by the user and the left electrode tip is installed on the left earpiece; and
the right earpiece comprises a right physical reference configured to rotationally constrain the right electrode tip within the ear canal of the user when the right earpiece is worn by the user and the right electrode tip is installed on the right earpiece.

4. The system of claim 3, wherein:
the left physical reference is configured to rest against a left concha of a left ear of the user when the left earpiece is worn by the user and the left electrode tip is installed on the left earpiece; and
the right physical reference is configured to rest against a right concha of a right ear of the user when the right earpiece is worn by the user and the right electrode tip is installed on the right earpiece.

5. The system of claim 2:
wherein:
the left electrode tip further comprises a left interface coupled to an inner surface of the left elastic substrate and configured to transiently engage a left earpiece; and
the right electrode tip further comprises a right interface coupled to an inner surface of the right elastic substrate and configured to transiently engage a right earpiece.

6. The system of claim 1, wherein:
the left earpiece houses a left in-ear audio speaker, the left earpiece and the left in-ear speaker configured as a left in-ear headphone; and
the right earpiece houses a right in-ear audio speaker, the right earpiece and the right in-ear speaker configured as a right in-ear headphone.

7. The system of claim 1, wherein the left driven ground electrode is electrically coupled to the right driven ground electrode to form a split driven ground electrode.

8. The system of claim 7, wherein the signal processing subsystem comprises a driven right leg circuit electrically coupled to the driven right leg electrode and configured to reduce common-mode interference in the left voltage differential and the right voltage differential.

9. The system of claim 8, wherein:
the left sense electrode defines a first surface area;
the right sense electrode defines the first surface area;
the left reference electrode defines the first surface area;
the right reference electrode defines the first surface area;
the left driven ground electrode defines a second surface area, the second surface area approximating half of the first surface area; and
the right driven ground electrode defines the second surface area.

10. The system of claim 1, further comprising a controller configured to classify a mental state of the user during the first period based on the left voltage signal and the right voltage signal.

11. The system of claim 1:
further comprising a controller configured to detect whether the left electrode tip is seated within an ear canal and whether the right electrode tip is seated within an ear canal during the first period based on the left voltage signal and the right voltage signal;
wherein, the signal acquisition subsystem is further configured to, during a second period, output a second left voltage signal representing potential difference between the left sense electrode and the left reference electrode in response to detecting that the left electrode tip is seated within an ear canal and that the right electrode tip is not seated within an ear canal.

12. An electrode tip for sensing in-ear electrical signals, comprising:
an elastic substrate defining an outer surface and an inner surface and configured to conform the outer surface against an internal surface of an ear canal of a user when the electrode tip is inserted into the ear canal of the user;
a sense electrode arranged on the outer surface of the elastic substrate;
a reference electrode arranged on the outer surface of the elastic substrate;
a driven ground electrode arranged on the outer surface of the elastic substrate; and
an interface coupled to the inner surface of the elastic substrate and configured to transiently engage an earpiece housing and expose:
a first electrical tab electrically coupled to the sense electrode;
a second electrical tab electrically coupled to the reference electrode; and
a third electrical tab electrically coupled to the driven ground electrode.

13. The system of claim 12, wherein:
the sense electrode, the reference electrode, and the driven ground electrode are radially offset on the outer surface of the elastic substrate;
the sense electrode is arranged facing upward when the electrode tip is inserted into the ear canal of the user; and
the reference electrode is arranged facing downward and backward when the electrode tip is inserted into the ear canal of the user.

14. The system of claim 12, wherein the elastic substrate further comprises:
a first lobe configured to project outward the outer surface of the elastic substrate at a location of the sense electrode on the outer surface of the elastic substrate;
a second lobe configured to project outward the outer surface of the elastic substrate at a location of the reference electrode on the outer surface of the elastic substrate; and
a third lobe configured to project outward the outer surface of the elastic substrate at a location of the driven ground electrode on the surface.

15. The system of claim 12, wherein:
the sense electrode defines a first surface area;
the reference electrode defines the first surface area; and
the driven ground electrode defines the first surface area.

16. The system of claim 12, wherein each of the sense electrode, the reference electrode, and the driven ground electrode comprises a conductive fabric.

17. The system of claim 12, wherein each of the sense electrode, the reference electrode, and the driven ground electrode comprise a conductive substrate applied to the outer surface of the elastic substrate.

18. The system of claim 12, further comprising a heartrate electrode arranged on the outer surface of the elastic substrate and proximal the superficial temporal blood vessels of the user when the electrode tip is inserted into the ear canal of the user.

19. A method for extracting in-ear electrical signals from a user, comprising:
during a first period:
receiving, from a left electrode tip in a bilateral output mode, a first left voltage signal representing potential difference between a left sense electrode, coupled to the left electrode tip, and a right reference electrode, coupled to a right electrode tip; and
receiving, from the right electrode tip in a bilateral output mode, a first right voltage signal representing potential difference between a right sense electrode, coupled to the right electrode tip, and a left reference electrode, coupled to the left electrode tip;
detecting whether the left electrode tip is seated within an ear canal and whether the right electrode tip is seated within an ear canal based on the first left voltage signal and the first right voltage signal; and
in response to detecting that the left electrode tip is seated within an ear canal and that the right electrode tip is not seated within an ear canal, during a second period:
transmitting a command to a signal acquisition subsystem, electrically coupled to the left electrode tip and the right electrode tip, the command instructing the signal acquisition subsystem to switch the bilateral output mode of the left electrode tip to a unilateral output mode of the left electrode tip; and
receiving, from the left electrode tip in the unilateral output mode, a second left voltage signal representing potential difference between the left sense electrode and the left reference electrode based on the second left voltage signal and the second right voltage signal.

20. The method of claim 19, further comprising, based on the second left voltage signal, classifying a mental state of the user during the second period.
